(19) United States
(12) Patent Application Publication
(10) Pub. No.: US 2003/0062040 A1
(43) Pub. Date: Apr. 3, 2003

(54) Face mask ventilation/perfusion systems and method

(76) Inventors: Keith G. Lurie, Minneapolis, MN (US); Todd M. Zielinski, Minneapolis, MN (US); Scott Edward Harder, Minneapolis, MN (US); Teresa Marie Gisch, Inver Grove Heights, MN (US); Jeff Waffensmith, Robinsdale, MN (US); Matt V. Leyden, St. Paul, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/966,945

(22) Filed: Sep. 28, 2001

(51) Int. Cl. A61M 16/00

(52) U.S. Cl. 128/203.11; 128/202.28

(57) ABSTRACT

A medical rescue system comprises a pressure responsive valve system that is configured to permit respiratory gases to be actively passed through the valve system and to prevent the flow of respiratory gases through the valve system until a threshold negative intrathoracic pressure is exceeded. The valve system further includes a swivel mount, and a ventilation tube is provided to be pivotally coupled to the swivel mount in the valve system. A headrest may also be provided to elevate and tilt the patient's head back and away from the patient's chest to assist in ventilating the patient. The communication device may be stored in the headrest to send communication signals to a remote receiver for emergency service.

### BACKGROUND OF THE INVENTION

This invention relates generally to the field of emergency medicine, and in particular to the field of cardio pulmonary resuscitation (CPR). More specifically, the invention relates to equipment and techniques to facilitate the performance of CPR.

A wide range of medical conditions may pose a significant threat to the heath and well being of individuals. Such conditions may include, for example, sudden cardiac arrest, loss of breath, heart attack or heart rhythm abnormality, and the like. Such conditions often need immediate medical attention to save the individual from serious injury or death.

The ability to rapidly identify a person's medical condition and to prescribe and administer appropriate remedies is often a critical factor for successful treatment. However, since most medical emergencies occur outside of medical facility, proper treatment is often limited by the ability to rapidly deploy appropriate medical personnel and equipment.

Although many individuals are trained in emergency produces, such as the performance of CPR, the effectiveness of the treatment may be limited by the equipment used (or lack of equipment) and the ability to communicate with (or even contact) medical or emergency personnel.

### BRIEF SUMMARY OF THE INVENTION

Hence, in some aspects this invention is related to equipment that may be used to facilitate proper treatment of such conditions while in the field. The equipment may also be used to facilitate communications with an emergency center so that medical personnel may be dispatched, and in some cases so that appropriate treatment may begin even before the arrival of certified medical personnel.

In one embodiment, the invention provides an exemplary medical rescue system that includes a pressure-responsive valve system. The valve system is configured to permit respiratory gases to be actively passed through the valve system and into the patient's lungs to ventilate the patient, and also to prevent the flow of respiratory gases through the valve system during decompression of the patient's chest until a threshold of negative intrathoracic pressure is exceeded. At this point, respiratory gases are permitted to flow through the valve system into the patient's lungs. In this way, the valve system may augment the extent and duration of the negative intrathoracic pressure when performing CPR, while also permitting proper patient ventilation. The valve system further includes a swivel mount. The valve system is configured to be used with a ventilation tube that may be pivotally coupled to the swivel mount of the valve system.

With such a configuration, the valve system and ventilation tube may be stored separately. When ready for use, the ventilation tube may be snapped into the swivel mount of the valve system. For example, the swivel mount may comprise a socket that is formed in a housing of the valve system, and the ventilation tube may include a ball that may be held within the socket. The valve system may also be coupled to a facial mask that is placed over the user's mouth and nose. When in place, the user may begin performing CPR on the patient by repeatedly compressing the patient's chest. Optionally, the patient's chest may be actively lifted in an alternating manner with chest compressions. During each recovery or decompression phase of the CPR cycle, the pressure responsive valve system prevents respiratory gases from entering the lungs until the threshold of negative intrathoracic pressure is exceeded. In this way, the amount of negative intrathoracic pressure is augmented to facilitate blood flow back to the chest cavity. Conveniently, a ventilation bag may be coupled to the ventilation tube so that the patient may be periodically ventilated through the ventilation tube. The swivel mount facilitates movement of the ventilation bag to permit easier operation and positioning of the ventilation tube. Of course, the patient could be ventilated in other ways, such as by manually blowing into the ventilation tube.

In another aspect, the medical rescue system may include a headrest that may be placed under the patient's neck to elevate and tilt the patient's head back away from the patient's chest to assist in ventilating the patient. Optionally, the headrest may include one or more compartments for storing various components of the medical rescue system. For example, the compartment may store the valve system and the ventilation tube.

In another aspect, at least one head strap may be wrapped around the patient's head and mask and used to pull the mask toward the patient's face. Optionally, the head strap may be configured to attach to the headrest to hold the headrest in the proper position while also securing the facial mask to the patient's face.

The headrest may also be used to store a variety of other components of the rescue system. For example, the headrest may include a power supply for powering various electrical components of the system. For instance, an electronic metronome may be stored within the headrest and powered by the power supply. The metronome may be used to facilitate a desired rhythm when performing chest compressions. The power supply may also be used to supply power to a communication device that may send communication signals to a remote receiver. Other items that may be stored in the headrest include a cooling package to cool the cervical region and the carotid arteries in an effort to regulate blood flow to the brain during cardio pulmonary resuscitation. Still another component may be a cervical spine collar, part of which is comprised of the head rest casing, that may be placed around a patient's neck to stabilize the spine during the performance of cardio pulmonary resuscitation.

The communication device may be configured to communicate with a variety of external systems. Conveniently, the communication device may be stored in the headrest as previously described. Alternatively, the communication device may be stored separately, such as in a carrying case that is used to store various pieces of equipment of the rescue system. In one embodiment, the communications device may be programmed to send communication signals to a transceiver, with the transceiver in turn being configured to establish a communication link with an emergency center system. As one example of such an embodiment, the communication device may be a radio frequency transmitter that transmits a signal to a transceiver that is located within the patient's home. The transceiver then dials 911 over a traditional telephone line or using cellular technology to contact a 911 center. The 911 center may then call appropriate medical personnel to have them dispatched to the patient. Since the transceiver is located at the person's residence, the transceiver may be patient specific and may be pre-programmed with various information such as the patient's name, address, medical facts, real time patient hemodynamics, ECG, respiratory parameters, and the like. In this way, a patient that needs medical attention may rapidly contact a 911 center simply by actuating the communications device when accessing the equipment of the rescue system. The relevant patient data would then be automatically transmitted to the 911 center. In one convenient aspect, the headrest may be provided with a sensor so that when it is opened, the communication signal is automatically transmitted to the transceiver.

In an alternative embodiment, a two-way communication transceiver may be provided in the headrest, or in a carrying case that holds the components of the rescue system. In this way, a 911 emergency number may be dialed directly from the patient, such as by using wireless technology. Further, by using a two-way communication link, information may be provided back from medical personnel. Such information may include, for example, instructions on how to perform CPR, control signals used to operate any of the components of the rescue system, and the like.

### BRIEF DESCRIPTION OF THE DRAWINGS

FIG. 1 is a perspective view of one embodiment of a valve system that is coupled to a facial mask and that includes a pivotal ventilation tube according to the invention.

FIG. 2 is a schematic cross sectional view of the valve system of FIG. 1 showing its operation when gases are ejected into the ventilation tube.

FIG. 3 illustrates the valve system of FIG. 2 showing the flow of respiratory gases exhaled by a patient.

FIG. 4 illustrates the valve system of FIG. 2 during the recovery phase of CPR.

FIG. 5 is a side view of one embodiment of a facial mask according to the invention.

FIG. 6 illustrates a strap arrangement and headrest that may be coupled to the mask of FIG. 5.

FIG. 7 is a side view of an alternative embodiment of a facial mask according to the invention.

FIG. 8 is a top view of another embodiment of a strap system and headrest that may be used with the facial mask of FIG. 7.

FIG. 9A illustrates one embodiment of a headrest having a compartment for holding various components of a rescue system according to the invention.

FIG. 9B illustrates the components of the rescue system of FIG. 9A when assembled.

FIG. 10 is a schematic diagram of a rescue system having a communication device according to the invention.

FIG. 11 is a schematic diagram of an alternative rescue system having a communication device according to the invention.

FIG. 12 is a perspective view of one embodiment of a rescue system when coupled to a patient according to the invention.

FIG. 13 is a perspective view of another embodiment of a rescue system prior to assembly, and showing various components that are stored in a head rest.

FIG. 14 illustrates the rescue system of FIG. 13 after assembly.

FIG. 15 is a perspective view of a further embodiment of a rescue system according to the invention.

FIG. 16 is a perspective view of another embodiment of a valve system according to the invention.

FIG. 17 is a cross sectional side view of the valve system of FIG. 16.

FIG. 18 is an exploded view of the valve system of FIG. 16.

FIG. 19 illustrates a cross sectional side view of another valve system according to the invention.

### DETAILED DESCRIPTION OF THE INVENTION

The invention provides various equipment and techniques to facilitate rescue procedures, including the performance of CPR, ventilation, defibrillation, and the like. The equipment and techniques are particularly useful in connection with rescue attempts outside of a medical facility.

One of the pieces of equipment may comprise a pressure-responsive valve system that is used in connection with CPR procedures. Such a valve system may be configured to permit respiratory gases to be injected through the valve system where they flow to the patient's lungs, without resistance. The valve system is also configured to permit the patient to exhale any respiratory gases, without resistance. Still further, the valve system may be configured to prevent the inflow of respiratory gases to the patient's lungs during a decompression or recovery phase of CPR where the patient's chest is actively lifted or allowed to expand due to its own resilience. In this way, the duration and extent of negative intrathoracic pressure is augmented to enhance the amount of blood flow back to the patient's chest. The valve system may be configured to open to permit gases to enter into the patient's lungs once a threshold negative intrathoracic pressure has been achieved. As such, the valve system may operate under principles similar to those described in U.S. Pat. Nos. 5,551,420; 5,692,498; 6,062,219; 5,730,122; 6,155,257; 6,234,916 and 6,224,562, the complete disclosures of which are herein incorporated by reference. Further, the valve system may be configured in other arrangements, including those described hereinafter.

The valve systems of the invention typically include a valve housing. In one embodiment, a ventilation tube is coupled to the valve housing such as that the ventilation tube may both rotate and pivot with respect to the valve housing. In this way, when a ventilation bag is coupled to the ventilation tube, it may rotate at 360° about a horizontal axis and pivot approximately 0 to about 70° about a vertical axis. Conveniently, a locking mechanism may be provided to lock the ventilation tube in place once properly positioned. To accomplish such features, the ventilation tube may include the ball that snap fits into a socket within the housing, or vice versa. Alternatively, a ball and socket connection may be provided between the valve housing and the facial mask. Still further, the components of the valve system, in part or their entirety, may be placed within the ball and socket joint itself.

In another aspect, the rescue system may include a headrest or pillow that may be placed under the patient's neck to elevate and tilt the head back and away from the patient's chest to aid in adequate ventilation during resuscitation efforts. The headrest may be used in combination with a face mask that in turn is connected to the valve system. The face mask may conveniently contain heads straps that wrap around the patient's head and pull the face mask toward the patient's face. In this way, an air tight seal is provided between the mask and the patient's face. The head straps may be made of any elastic material, a hook and loop fastener material, such as Velcro™, and the like, which permit easy application and removal for the rescuer. The head strap may be secured to the face mask with plastic loops, similar to a belt loop on a pair of pants, and/or with channels. The head strap may also be incorporated into and attachable to the headrest. In this way, the straps not only couple the face mask to the patient's face but also hold the headrest in the proper position to elevate and tilt the head back and away from the patient's chest.

The headrest may also include a storage compartment to house the various components of the rescue system, including the facial mask, head straps, ventilation tube, and the like. Other features that may be stored in the compartment include an audio/visual metronome, communications circuitry, electronic stimulation electrodes, and stimulation components for phrenic nerve stimulation of the diaphragm, a defibrillator, and the like. Other components that may be included include a thermal cooling chemical ice package to cool the cervical region and primarily the carotid arteries in an effort to regulate blood flow to the brain during CPR. Moreover, a power supply, such as a battery, may be housed within the compartment to provide power to the various electrical components of the rescue system. Another piece of equipment that may be stored within the compartment is a cervical spine collar or “C collar” for trauma induced injuries. The C collar is placed around the patient's neck to stabilize the spine during the performance of CPR, and may utilize the case of the storage compartment as part o the stabilizing aspect of the C collar. In some embodiments, the headrest itself can be used as part of the C-collar to support and stabilize the neck.

As previously described, one component of the rescue system may be electronic metronome to facilitate the timing of chest compressions during the performance of CPR. The electronic metronome may be incorporated into the facial mask, or stored in the headrest. One example of an electronic metronome that may be used is described in co-pending U.S. patent application Ser. No. 09/532,601, filed Mar. 22, 2000, the complete disclosure of which is herein incorporated by reference.

Another feature of the rescue system is that in one or two-way communications circuitry may be provided to permit the rescuer to communicate with the remote system, such as a 911 system, a medical facility, or the like. For example, the rescue system may include the transmitter that is configured to communicate with a remote receiver unit which in turn may communicate with a central 911 emergency dispatching center or a medical facility. Alternatively, the rescue system may include a transceiver which is able to transmit and receive communications to and from a 911 emergency dispatching center, a medical facility, or the like. The circuitry may include various pre-programmed information that is to be transmitted, such as the patient's name, address, medical facts, real time patient hemodynamics, ECG, respiratory parameters, and the like. In this way, the rescue system may be used in combination with a known group of people, such as within a household. When a medical emergency arises, the pre-programmed information may be for those residing at the household and may be automatically transmitted to a 911 center or medical facility. The communication system may be actuated by a switch that is turned on by the user or automatically actuated when the storage compartment of the headrest is opened to retrieve the resuscitation devices. Signals may be transmitted on a 900 MHz or 2.4 Hz center frequency using either standard cellular telephone technology or Wireless Personal Area Network (WPAN) technology (Bluetooth or IEE 802.11b). Conveniently, a rechargeable battery may be used to permit the unit to be plugged into a 110 AVC wall outlet. The user may view a charge meter located on the external housing of the device to determine if the battery is fully charged. Circuitry for a “low charge” battery may be incorporated into the system as a safety feature to produce an audible beep indicating that the unit should be recharged.

One exemplary embodiment uses a small transmission device in the storage compartment of the headrest or other carrying case to act as a transmit only device to transmit an activation signal to a remote transceiver device. The remote device is then capable of dialing 911 either through a land line telephone or cellular telephone technology after receiving the activation signal. Once the wireless connection has been made, patient data (either programmed or acquired) may be transmitted to the applicable receiver site. Technology for transmitting such signals is described in U.S. Pat. No. 5,971,921, the complete disclosure of which is herein incorporated by reference.

Another embodiment may incorporate all transmission devices in the headrest or an associated carrying case. Hence, when a resuscitation attempt is made, the rescuer simply opens the compartment, turns on an activation switch (or automatic activation may occur when the compartment is accessed) that dials 911 using cellular telephone technology that may be programmed with pre-determined information. Alternatively, a two-way, hands free (speak phone) communications link between the 911 operator and the rescuer may be established. Communication links may then be routed by the 911 operator to medically trained personnel to provide guidance to the rescuer up until the emergency medical team arrives. Once the wireless connection has been made, patient data (either programmed or acquired) may be transmitted to the applicable receiver site. Further, signals from the medical facility may be transmitted back to components of the rescue system in a manner similar to that described in co-pending U.S. patent application Ser. No. 09/564,889, filed May 4, 2000, the complete disclosure of which is herein incorporated by reference.

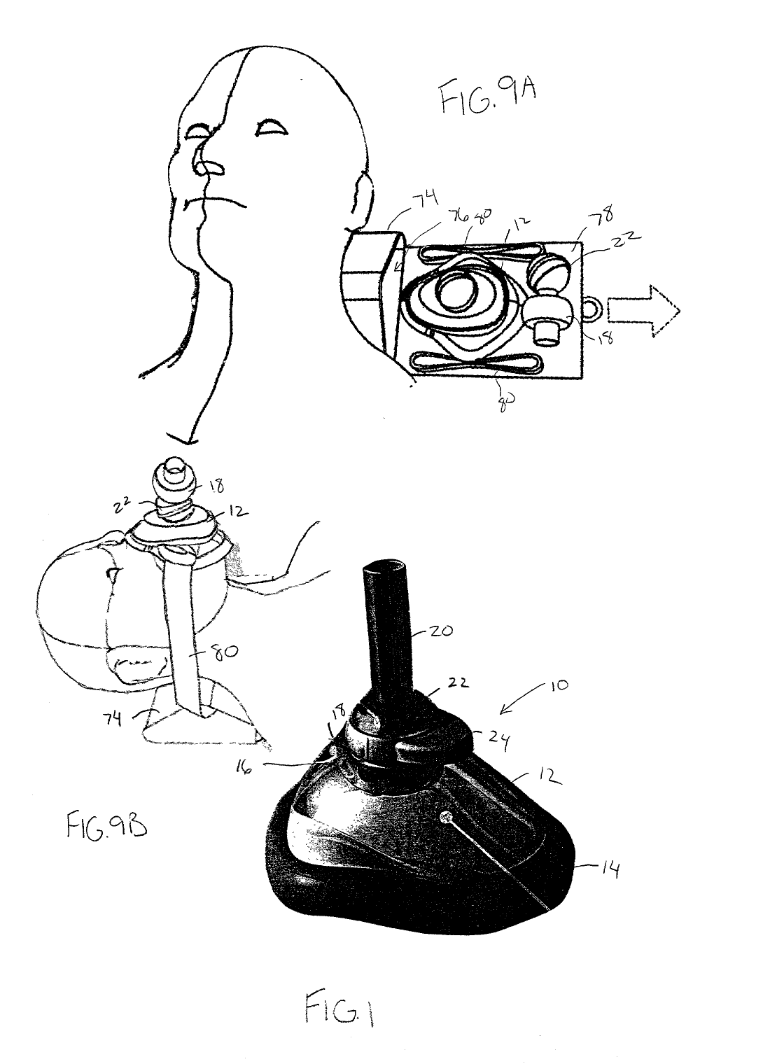

Referring now to FIG. 1, one embodiment of a face mask ventilation system 10 will be described. System 10 includes a facial mask 12 having a gel filled, air filled, foam filled, or silicone membrane/interface seal 14 that conforms to the patient's face. Optionally, the form fitting mask-to-face interface may include flexible metal strips that when compressed to the morphology of the patient's face, retain their shape and conform the interface material to the specific shape of the patient's face. For instance, a flexible metal aluminum strips may be embedded into the mask to conform to the shape of the face. As another option, the mask may include a secondary seal mechanism to conform to the patient's mouth and nose. Face mask 12 includes another interface seal 16 for coupling with a pressure responsive valve system 18. As described hereinafter, valve system 18 is used to enhance the extent and duration of negative intrathoracic pressure when performing CPR. Rotationally and pivotally coupled to valve system 18 is a ventilation tube 20. Ventilation tube 20 includes a ball 22 at its distal and that is received within a corresponding socket of valve system 18. In this way, ventilation tube 20 may rotate 360° about a horizontal axis and to pivot about 70° relative to a vertical axis. In this way, when a ventilatory bag or other ventilation structure is coupled to ventilation tube 20, it may be positioned at a wide variety of orientations to facilitate ventilation of the patient. Conveniently, a lock 24 may be used to lock ventilation tube 20 in a particular orientation.

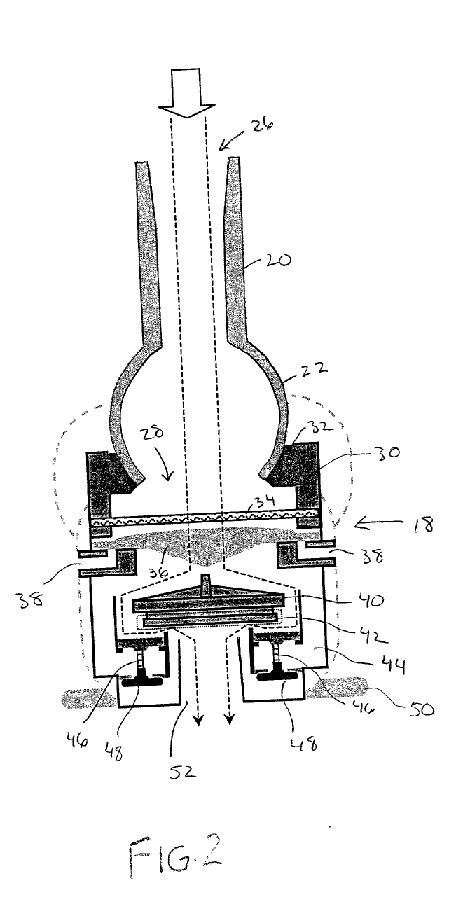

Referring now to FIGS. 2-4, operation of valve system 18 will be described schematically. For convenience of discussion, the same reference numerals used to describe the face mask ventilation system 10 of FIG. 1 will also be used in connection with FIGS. 2-4. As previously mentioned, ventilation tube 20 may rotate and pivot relative to valve system 18. Ventilation tube 20 includes a ventilation bag port 26 where a ventilation bag may be coupled to tube 20. Ball 22 also includes a port 28 to permit respiratory gases to flow through ventilation tube 20.

Valve system 18 includes a valve housing 30 with a socket 32 into which ball 22 is received. In this way, ventilation tube 20 may rotate about a horizontal axis and pivot relative to a vertical axis as previously described. Disposed in valve housing 30 is a filter 34 that is spaced above a duck bill valve 36. Duck bill valve 36 also rests upon expiratory gas ports 38. Duck bill valve 36 is spaced above a diaphragm holder 40 which holds a diaphragm 42. Valve system 18 further includes a gasp inspiratory port 44 and a check valve recoil spring 36 that is coupled to a check valve 48. Conveniently, check valve 48 comprises a rubber seal. Further, a face mask locking flange 50 is provided to mate with interface seal 16 of mask 12. Valve system 18 further includes an exit port 52 that may exit into mask 12 or may alternatively be coupled to an endotracheal tube.

When ventilation tube 12 and valve system 18 are coupled to a facial mask, which in turn is coupled to a patient, CPR may be performed on the patient. This may be traditional CPR, active compression/decompression CPR, vest CPR, and the like as generally described in U.S. Pat. Nos. 5,551,420; 5,692,498; 6,062,219; 5,730,122; 6,155,257; 6,234,916 and 6,224,562 previously incorporated by reference. Shown in FIG. 2 is the gas flow when the patient is actively ventilated by forcing respiratory gases through ventilation tube 20 as illustrated by the arrow. The gas flows through filter 34, through duck bill valve 36, and forces up diaphragm 42 to permit the gases to exit through exit port 52. Hence, at any time during the performance of CPR the patient may be ventilated simply by forcing the respiratory gases through tube 20.

FIG. 3 illustrates gas flows expired by the patient which typically occurs during the compression phase of CPR. The expired gases flow through exit port 52 as illustrated by the arrow and lift up diaphragm 42. The gas flow then lifts up duck bill valve 36 and exists through expiratory gas ports 38 as shown.

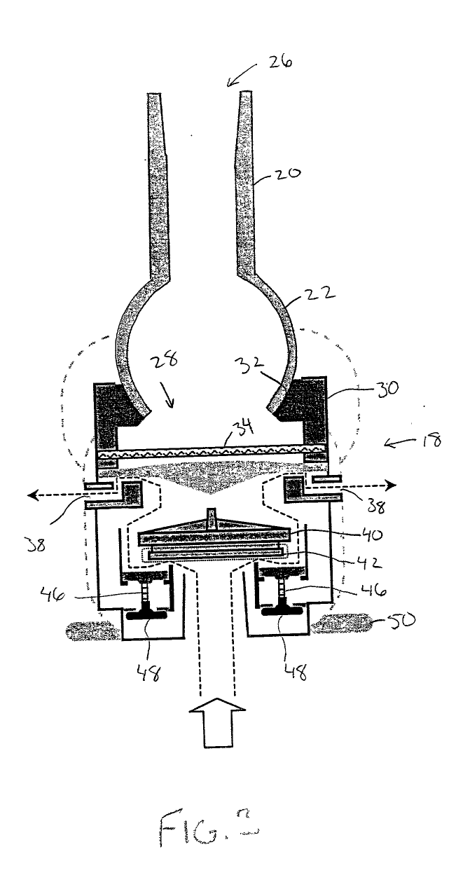

FIG. 4 illustrates gas flows gas flows during the recovery or decompression phase of CPR where the patient's chest may be actively lifted. In so doing, valve system 18 prevents respiratory gases from flowing into the lungs until a threshold of negative intrathoracic pressure level is exceeded. When this pressure level is exceeded, trip valve 48 is pulled downward to permit respiratory gases to flow through check valve and to the patient's lungs by initially passing through tube 20 and duck bill valve 36 as shown. Valve 48 may be set to open when the negative intrathoracic pressure is in the range from about −5 cm H₂O to about −30 cm H₂O. These parameters may vary depending on a variety of factors including the condition being treated, patient size, and the like. Hence, the magnitude and duration of negative intrathoracic pressure may be enhanced during decompression of the patient's chest by use of valve system 18. Once the intrathoracic pressure falls below the threshold, recoil springs 46 again close check valve 48. The valve may be connected to a variety of systems or devices to ventilate the patient. These include, for example, a resuscitator bag, such as those commercially available from Ambu International, a mouth to valve tube, an automatic ventilator, and/or an oxygen source, and the like. The valve may also include a port to lead to an end tidal CO₂ monitor and connections to measure oxygen, pressure within the body and other physiological parameters. The valve may also have an attached valve to create a range of positive end expiration pressures (PEEP) that typically occurs during compression of the patient's chest. This valve may be set to open when the intrathoracic pressure is in the range from about 0 cm H₂O to about 20 cm H₂O.

Referring now to FIG. 5, one alternative embodiment of a facial mask 54 will be described. Mask 54 includes a gel or air filled membrane/interface seal 56 similar to that previously described with FIG. 1. Mask 54 also includes an interface seal 58 where a pressure responsive valve system 18 may be coupled in a manner similar to that previously described with FIG. 1. Mask 54 further includes a pair of head strap loops 60.

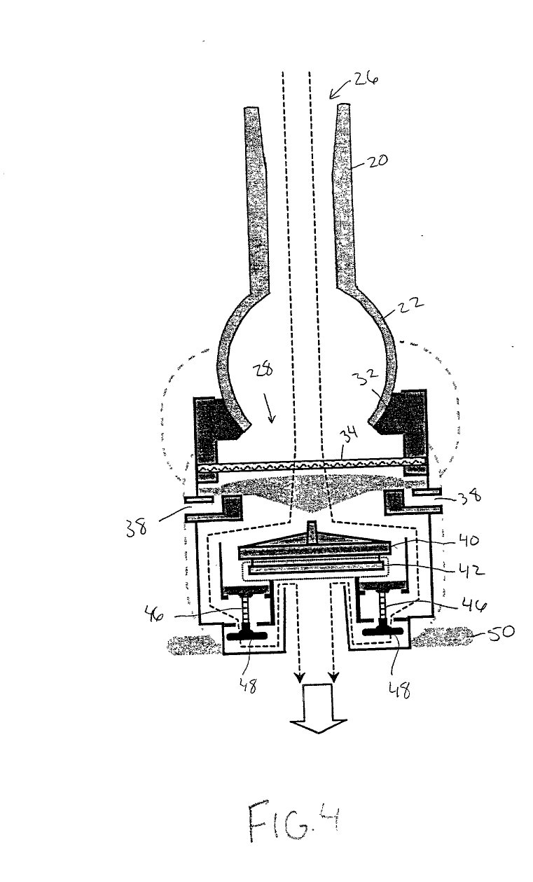

FIG. 6 illustrates a strap system 62 that may be used with mask 54. Strap system 62 includes a pair of straps 64 that pass through loops 60 to secure strap system 62 to mask 54. Straps 64 includes a securing material 66, such as a hook-and-loop fastener material, commonly referred to as Velcro™. Material 66 is configured to be secured to other securing material 68 at the opposite end of the strap system. Further, straps 64 define an opening 70 that permits straps 64 to be positioned around the valve system when coupled to mask 54. Hence, in operation straps 64 are placed through loops 60 and material 66 is secured to material 68 after passing around the patient's head.

Strap system 62 further includes a neck pillow or headrest 72 that is configured to be positioned underneath the patent's neck to tilt the head backward when performing CPR. Straps 64 further facilitate the positioning and holding of headrest 72 against the patient's neck while also securing facial mask 54 to the patient's face.

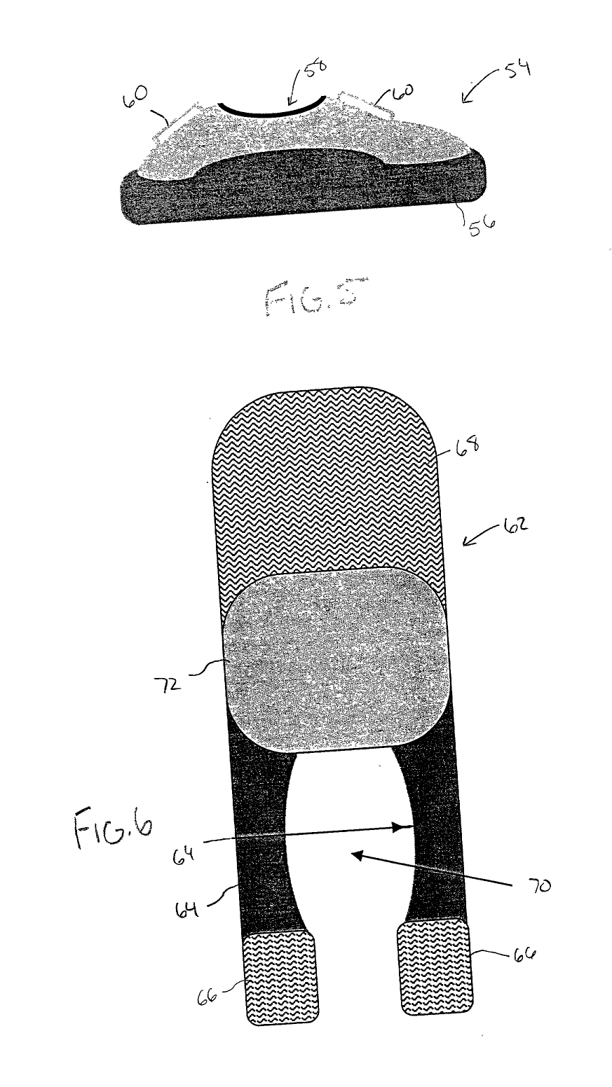

FIGS. 7-8 illustrate alternative embodiments of facial mask and strap systems. For convenience of discussion, identical elements will be referred to with the same reference numerals used in connection with FIGS. 5-6. Modified elements will be referred to with the same reference numerals followed by a “′”. Hence, in FIG. 7, mask 54′ differs from mask 54 in that mask 54′ includes channels 61 instead of loops 60. Further, in FIG. 8 material 66′ extends between both straps 64. In operation, straps 64 fit within channels 61 and strap system 62 is placed around the patient's head to secure face mask 54′ to the patient's face while also securing headrest 72 beneath the patient's neck.

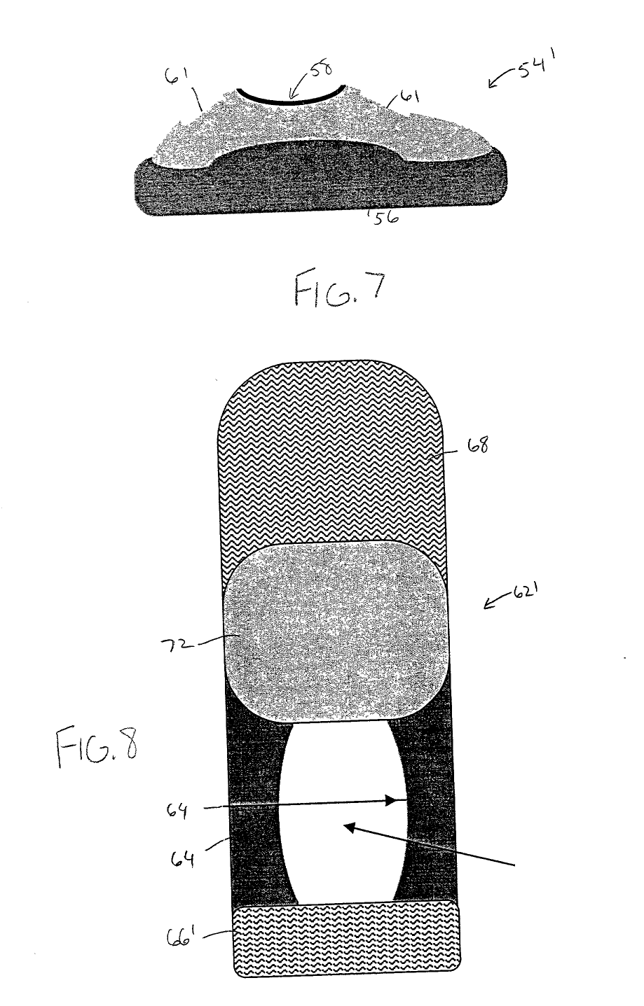

Referring now to FIG. 9A, one embodiment of a headrest 74 that may be used with any of the systems set forth herein will be described. Headrest 74 includes a storage compartment 76 to house the various components of the face mask ventilation system. Conveniently, these components may be held on a drawer 78 that may be pulled out of storage compartment 76 as shown by the arrow. As shown, drawer 78 holds face mask 12, pressure responsive valve system 18, ball 22 of ventilation tube 20 and a pair of head straps 80. However, it will be appreciated that any components of a rescue system may be stored in storage compartment 76, and the invention is not intended to be limited to the specific items illustrated in FIG. 9. For example, storage compartment 76 may be employed to store an audio/visual metronome, two-way communications circuitry, electronic stimulation electrodes, stimulation components for phrenic nerve stimulation of the diaphragm, physiological monitors or transducers for measuring oxygen, carbon dioxide, temperature, and the like. Other components that may be stored include a thermal cooling chemical ice package to cool the cervical legion, a power supply, such as a battery package, a cervical spine collar, and the like. As described hereinafter, headrest 74 may also include a sensor to determine when drawer 78 is withdrawn. This may then actuate the communication circuitry to dial a 911 emergency center. Details of an electric metronome that may be used are described in co-pending U.S. patent application Ser. No. 09/532,601, filed Mar. 22, 2000, and details of equipment for electronic stimulation electrodes are described in co-pending U.S. patent application Ser. No. 09/315,396, filed May 20, 1999 and Ser. No. 09/533,880, filed Mar. 22, 2000, the complete disclosures of which are herein incorporated by reference.

In use, headrest 74 may be placed beneath the patient's neck as illustrated in FIG. 9A. Drawer 78 may be opened to access the various components of the rescue system which may then be assembled and coupled to the patient as shown in FIG. 9B. CPR may then be performed on the patient.

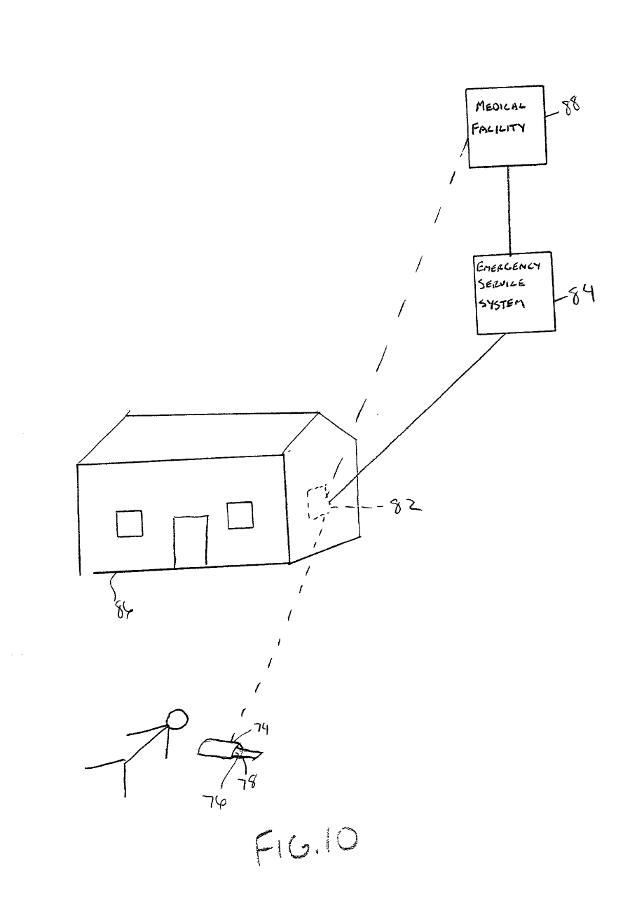

Referring now to FIG. 10, a communication system for communicating with emergency equipment or personnel will be described. Conveniently, the system may utilize headrest 74 which includes a small transmission device that is stored in storage compartment 76 and acts as a transmit only device to transmit an activation signal a remote transceiver device 82. The activation signal may be produced when drawer 78 is opened or when a switch is manually activated on headrest 74. Transceiver device 82 is then configured to dial an emergency service system 84 over a traditional telephone line or using cellular telephone technology. The emergency service system 84 may be contacted by dialing a 911 telephone number as is known in the art.

One advantage of transceiver device 82 is that it may be programmed with information specific to certain individuals, such as those that live within house 86. In this way, once the connection is made with emergency service system 84, the pre-programmed data may automatically be transmitted to emergency service system 84. Such information may include the patient's name, address, medical facts, real time patient hemodynamics, ECG, respiratory parameters, and the like. Alternatively, data acquired while treating the patient may also be transmitted from transceiver device 82 to emergency service system 84.

Emergency service system 84 may then dispatch appropriate medical personnel to the patient. Emergency service system 84 may also contact a medical facility 88 to notify them of the emergency. Optionally, medical information may be transmitted from either emergency service system 84 or medical facility 88 to transceiver 82 to facilitate the rescue attempts. Optionally, as shown in dashed line transceiver 82 may also be programmed to communicate directly with medical facility 88.

Conveniently, headrest 74 may include the rechargeable battery that may be recharged using a 110 VAC wall outlet. The charge meter may be located on the exterior of headrest 74 to indicate when the battery needs to be recharged. Further, an audio beep may also be produced indicating that a recharge is needed.

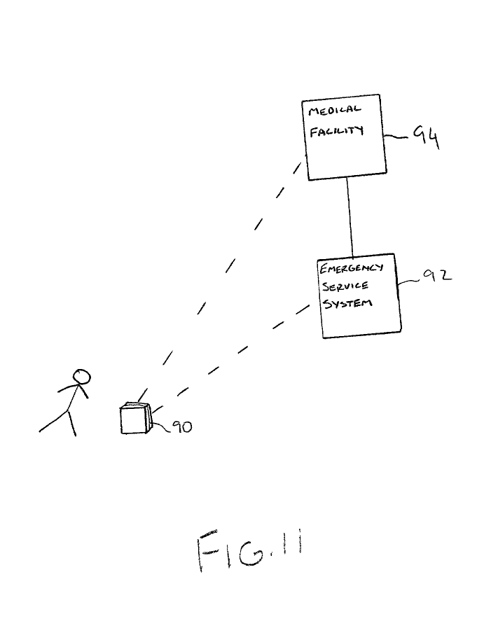

An alternative design of a communication system is described in FIG. 11. In FIG. 11, a transceiver device is held within a carrying case 84 (or alternatively in the storage compartment of a headrest). When a resuscitation attempt is required, the rescuer may simply open carrying case 90 or access the storage compartment in the headrest and turn on an activation switch (or automatic activation may occur) that dials a 911 number utilizing cellular telephone technology that is pre-programmed with predetermined information to establish a two-way, hands free (using a speaker telephone) communications link between an emergency service system 92 and the transceiver. Communications may then be routed by the 911 operator of emergency service system 92 to medically trained personnel to provide guidance to your rescuer until the emergency medical team arrives. Further, a link may be established with a medical facility 94 that may also provide communications directly to the transceiver. Conveniently, patient data (either programmed or acquired) may be transmitted back and forth between the transceiver and either emergency service system 92 or medical facility 94. For example, the rescue system may include various monitoring devices or electrical stimulation devices that may be controlled directly from medical facility 94 in a manner similar to that described in co-pending U.S. patent application Ser. No. 09/564,889, filed May 4, 2000, previously incorporated herein by reference.

Hence, by using the system of FIG. 11, a rescuer may rapidly establish a two-way communications link between a 911 service or between a medical facility or information regarding the patient may be transferred to the medical facility for 911 service while instructions may be transmitted to the rescuer. Further, monitoring signals may be sent from the patient to a medical facility for 911 service while control signals to operate various rescue equipment may be transmitted from the medical facility or the 911 emergency service.

FIG. 12 illustrates another embodiment of a rescue system 100. Rescue system 100 comprises a valve system 102 that may be similar to any of the valve systems described herein. Valve system 102 is coupled to a facial mask 104 that is coupled to a head strap 106 that holds mask 104 to the patient's face. Head strap 106 is also configured to extend along the back of the patient's head and is pivotally coupled to a head rest 108 to tilt the head backward when performing CPR.

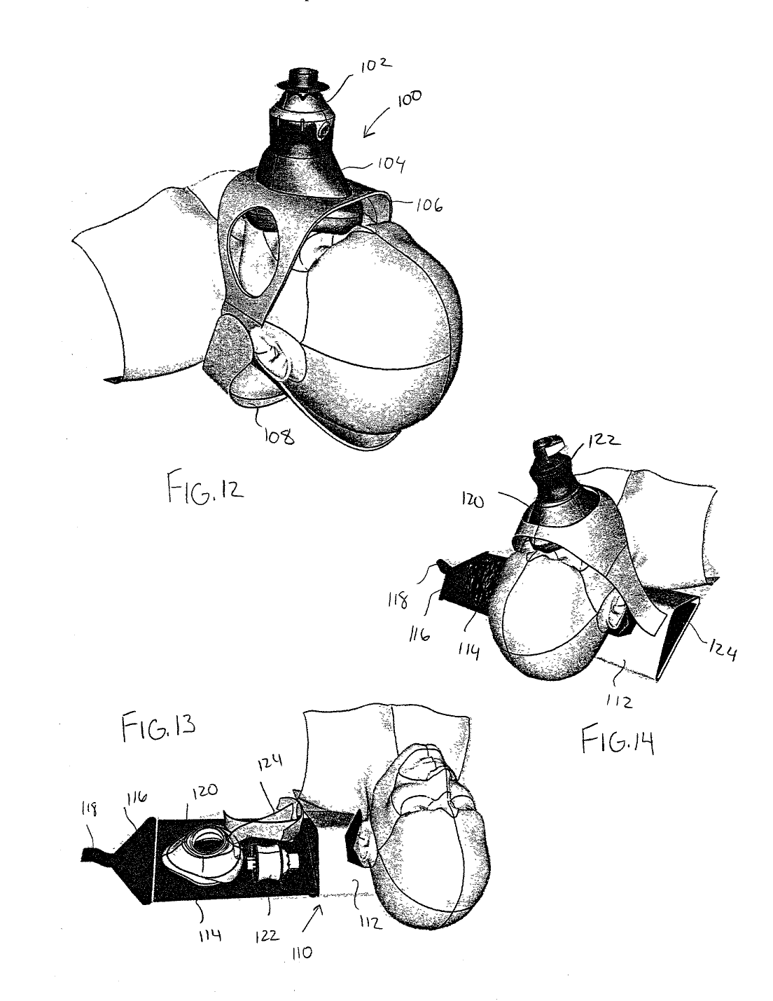

FIG. 13 illustrates another embodiment of a rescue system 110 prior to assembly. System 110 comprises a head rest 112 that forms a compartment for holding various components of the system. The components are conveniently stored on a platform 114 that slides in an out of head rest 112. A side 116 is coupled to platform 114 to enclose the components within the compartment during storage. Hence, to access the components, a tab 118 is pulled to open side 116 and to slide platform 114 from the compartment. Shown on platform 114 is a facial mask 120 and a valve system 122 that may be similar to any of those described herein. A head strap 124 is also provided on platform 114. Although not shown, it will be appreciated that any of the other components described herein may also be stored in the compartment.

As shown in FIG. 14, head rest 112 is placed under the patient's head to tile the head backward. After platform 114 is withdrawn, valve system 122 is attached to mask 120 and head strap 124 is used to couple mask 120 to the patient's face. Conveniently, strap 124 connects to head rest 112 using a fastener, such as a hook and loop fastener material.

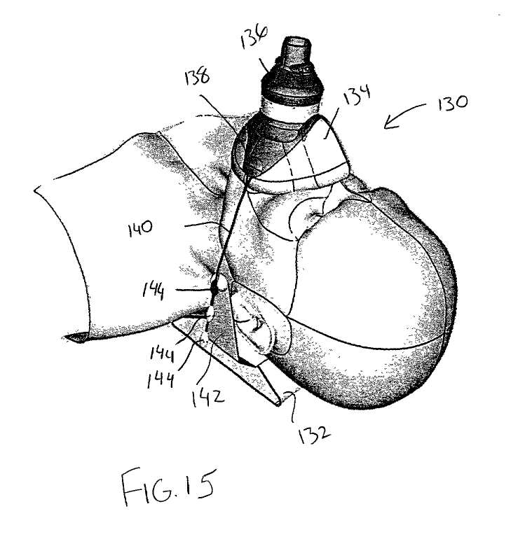

FIG. 15 illustrates a further embodiment of a rescue system 130. The rescue system 130 includes a head rest 132 for tilting the head backward. Head rest 132 may optionally include a compartment for holding various components of the rescue system. The rescue system 130 further includes a facial mask 134 that is coupled to a valve system 136 that may be similar to any of those described herein. Mask 134 also includes tabs 138 for coupling cords 140 to mask 134. Head rest 132 also includes a flange 142 to permit the other ends of cords 140 to be attached. Conveniently, cords 140 may include stops 144 to permit the distance between mask 134 and head rest 132 to be adjusted. In this way, the pressure applied to the patient's face by mask 134 may be adjusted to provide an appropriate seal.

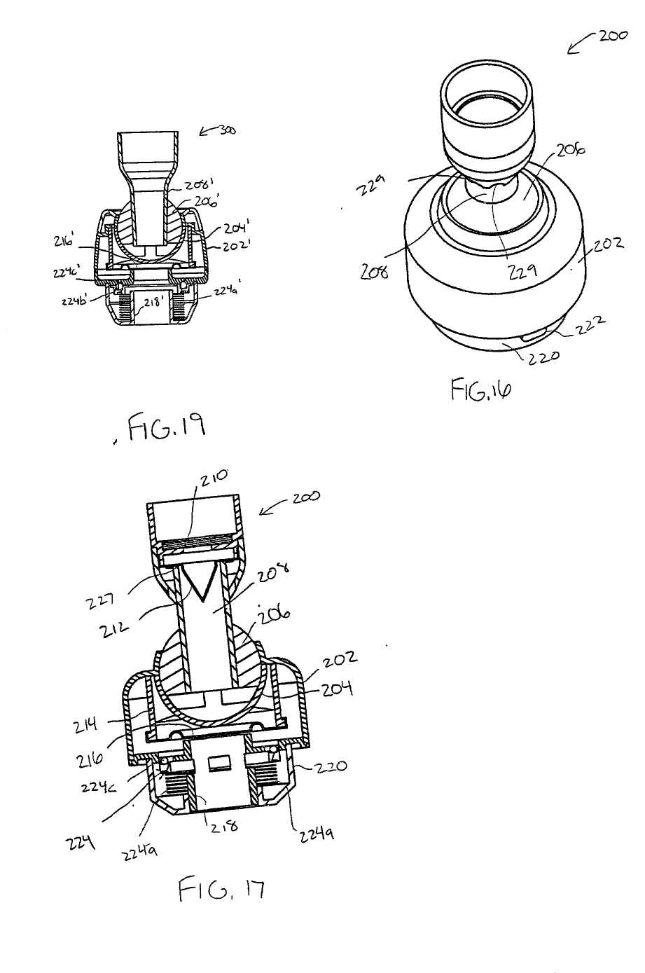

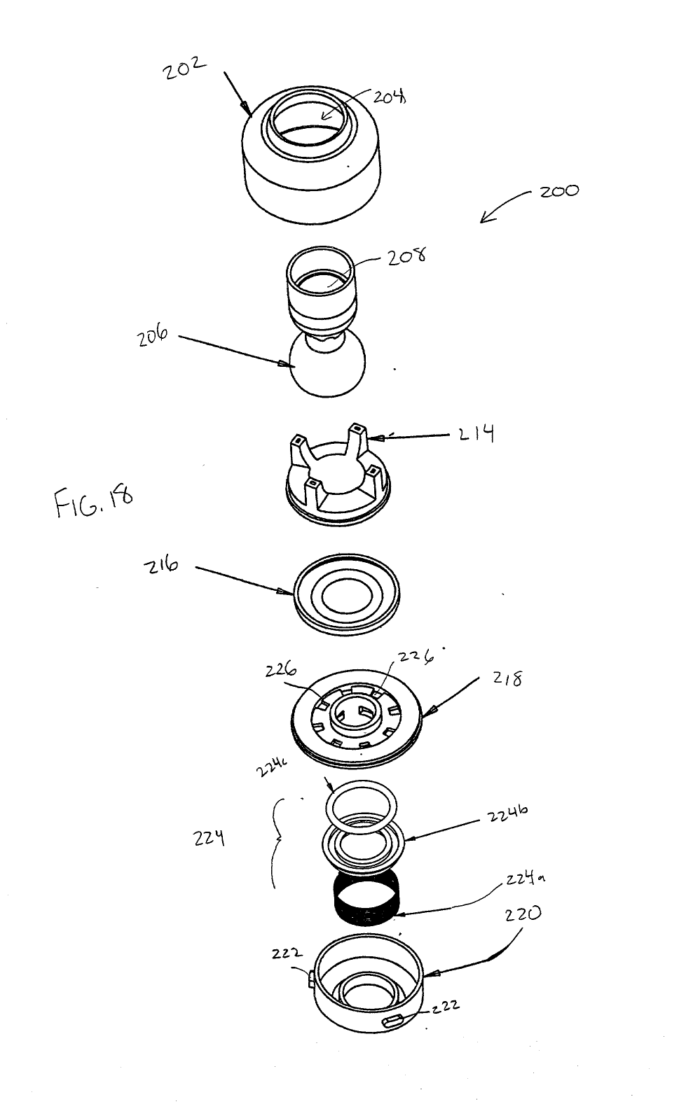

FIGS. 16-18 illustrate another embodiment of a valve system 200 that may be coupled to a facial mask, an endotracheal tube, or the like as well as to a ventilatory source. Valve system 200 includes a valve housing 202 with a socket 204 into which a ball 206 of a ventilation tube 208 is received. In this way, ventilation tube 208 may rotate about a horizontal axis and pivot relative to a vertical axis in a manner similar to other embodiments. Disposed in ventilation tube 208 is a filter 210 that is spaced above a duck bill valve 212. A diaphragm holder 214 that holds a diaphragm 216 is held within housing 202. Valve system 200 further includes a patient port 218 that is held in place by a second housing 220. Housing 220 conveniently includes tabs 222 to facilitate coupling of valve system 200 with a facial mask. Also held within housing 220 is a check valve 224 comprising a spring 224a, a ring member 224b and an o-ring 224c. Spring 224a biases o-ring against patient port 218. Patient port 218 includes bypass openings 226 that are covered by o-ring 224c until the pressure in patient port 218 reaches a threshold negative pressure to cause spring 224a to compress.

When the patient is actively ventilated, respiratory gases are forced through ventilation tube 208. The gases flow through filter 210, through duck bill valve 212, and forces up diaphragm 214 to permit the gases to exit through port 218. Hence, at any time during the performance of CPR the patient may be ventilated simply by forcing the respiratory gases through tube 208.

During the compression phase of CPR, expired gases flow through port 218 and lift up diaphragm 214. The gases then flow through a passage 227 in ventilation tube 208 where they exit the system through openings 229 (see FIG. 16).

During the recovery or decompression phase of CPR where the patient's chest is actively lifted, valve system 200 prevents respiratory gases from flowing into the lungs until a threshold of negative intrathoracic pressure level is exceeded. When this pressure level is exceeded, ring member 224b of check valve 224 is pulled downward as spring 224a are compressed to permit respiratory gases to flow through openings 226 and to the patient's lungs by initially passing through tube 208 and duck bill valve 212. Valve 224 may be set to open when the negative intrathoracic pressure is in the range from about −5 cm H₂O to about −30 cm H₂O. These parameters may vary depending on a variety of factors including the condition being treated, patient size, and the like. Hence, the magnitude and duration of negative intrathoracic pressure may be enhanced during decompression of the patient's chest by use of valve system 200. Once the intrathoracic pressure falls below the threshold, recoil spring 224a again closes check valve 224.

Shown in FIG. 19 is an alternative embodiment of a valve system 300 that is similar to valve system 200. For convenience of discussion, similar elements are referred to with the same reference numeral, followed by a “′”. Valve system 300 operates in a manner similar to valve system 200 except that valve system 300 does not include a duck bill valve. As such, expired gases flow back through ventilation tube 208′. All other functions are similar to valve system 200.

The invention has now been described in detail for purposes of illustration and example. However, it will be appreciated that certain modifications may be made within the scope of the appended claims.

What is claimed is:

1. A medical rescue system, comprising:
a pressure responsive valve system that is configured to permit respiratory gases to be actively passed through the valve system and to prevent the flow of respiratory gases through the valve system until a threshold negative intrathoracic pressure is exceeded, wherein the valve system further includes a swivel mount; and
a ventilation tube that is adapted to be pivotally coupled to the swivel mount of the valve system.

2. A system as in claim 1, wherein the valve system includes a valve housing, wherein the swivel mount comprises a socket formed in the valve housing, and wherein the ventilation tube includes a ball that is configured to be held within the socket.

3. A system as in claim 1, further comprising a facial mask, and wherein the valve system is configured to be coupled to the facial mask.

4. A system as in claim 1, wherein the valve system further comprises a one way valve that permits air to be forced through the valve system and to a patient, and a pressure responsive valve that opens once the threshold negative intrathoracic pressure is exceeded to permit air to flow through the valve system and to the patient.

5. A system as in claim 1, further comprising a head rest that is adapted to be placed under a patient's neck to elevate and tilt the patient's head back away from the patient's chest.

6. A system as in claim 2, wherein the head rest includes a compartment that is adapted to store the valve system and the ventilation tube.

7. A system as in claim 1, further comprising at least one head strap that is adapted to be wrapped around the patient's head and the mask to pull the mask toward the patient's face.

8. A system as in claim 1, wherein the valve system further includes a positive end expiratory pressure valve to prevent respiratory gases from exiting the lungs until a threshold positive intrathoracic pressure is exceeded, and wherein the threshold pressure is in the range from about 0 cm H₂O to about 20 cm H₂O.

9. A method for coupling a rescue system to a patient, the method comprising:
providing a pressure responsive valve system that is configured to permit respiratory gases to be actively passed through the valve system and to the patient's lungs, and to prevent the flow of respiratory gases through the valve system and to the patient's lungs until a threshold negative intrathoracic pressure is exceeded, wherein the valve system further includes a swivel mount;
pivotally coupling a ventilation tube to the swivel mount of the valve system; and
interfacing the valve system with the patient's airway.

10. A method as in claim 9, wherein the valve system is interfaced with the patient's airway by attaching a facial mask over the patient's mouth and nose, and wherein the valve system is coupled to the facial mask.

11. A method as in claim 9, wherein the valve system includes a valve housing, wherein the swivel mount comprises a socket formed in the valve housing, and wherein the ventilation tube includes a ball that is inserted into the socket.

12. A method as in claim 9, further comprising positioning a head rest under the patient's neck to elevate and tilt the patient's head back away from the patient's chest to assist in ventilating the patient.

13. A method as in claim 12, wherein the head rest includes a compartment wherein the valve system and the ventilation tube are stored, and further comprising removing the valve system and the ventilation tube from the compartment prior to their coupling.

14. A method as in claim 10, further comprising wrapping at least one head strap around the patient's head and the mask to pull the mask toward the patient's face.

15. A method as in claim 9, further comprising performing CPR on the patient.

16. A method as in claim 9, further comprising actively forcing respiratory gases through the tube and into the patient's lungs.

17. A rescue system, comprising:
a head rest that is adapted to be placed under a patient's neck to elevate and tilt the patient's head back away from the patient's chest to assist in ventilating the patient;
a pressure responsive valve system that is configured to permit respiratory gases to be actively passed through the valve system and to prevent the flow of respiratory gases through the valve system until a threshold negative intrathoracic pressure is exceeded; and
a facial mask that is adapted to be placed over the patient's mouth and nose;
wherein the valve system is adapted to be coupled to the facial mask, and wherein the facial mask is configured to be operably coupled to the head rest.

18. A system as in claim 17, further comprising a strap coupled to the head rest that is adapted to be coupled to the facial mask.

19. A system as in claim 17, wherein the head rest includes a compartment that is adapted to hold the valve system and the facial mask.

20. A system as in claim 19, further comprising an electronic metronome that is adapted to be stored in the compartment.

21. A system as in claim 19, further comprising a power supply that is adapted to be stored in the head rest.

22. A system as in claim 19, further comprising a communication device that is adapted to be stored in the compartment, wherein the communication device is adapted to send communication signals to a remote receiver.

23. A system as in claim 19, further comprising a cooling package that is adapted to be stored in the compartment.

24. A system as in claim 19, further comprising a cervical spine collar that is adapted to be stored in the compartment.

25. A system as in claim 17, wherein the valve system further includes a positive end expiratory pressure valve to prevent respiratory gases from exiting the lungs until a threshold positive intrathoracic pressure is exceeded, and wherein the threshold pressure is in the range from about 0 cm H₂O to about 20 cm H₂O.

26. A system as in claim 19, further comprising an end tidal carbon dioxide detector coupled to the valve system and in communication with a monitor that is adapted to be stored in the compartment.

27. A method for rescuing a patient, the method comprising:
placing a head rest under a patient's neck to elevate and tilt the patient's head back away from the patient's chest to assist in ventilating the patient; and
coupling a facial mask over the patient's mouth and nose and to the head rest, wherein the facial mask includes a pressure responsive valve system that is configured to permit respiratory gases to be actively passed through the valve system and to prevent the flow of respiratory gases through the valve system until a threshold negative intrathoracic pressure is exceeded; and
repeatedly compressing the patient's chest, and allowing the chest to recoil after each compression.

28. A method as in claim 27, further comprising periodically forcing respiratory gases through the valve system to ventilate the patient.

29. A method as in claim 27, further comprising coupling the head rest to the facial mask with a strap.

30. A method as in claim 27, wherein the head rest includes a compartment where the valve system and the facial mask are held, and further comprising accessing the compartment and removing the valve system and the facial mask from the compartment.

31. A method as in claim 30, further comprising actuating an electronic metronome that is stored in the compartment.

32. A method as in claim 27, further comprising actuating a communication device to send communication signals to a remote receiver.

33. A method as in claim 30, further comprising providing a cooling package in the head rest, and using the cooling package to lower the temperature of the head rest.

34. A method as in claim 30, further comprising removing a cervical spine collar that is stored in the compartment and placing the collar about the patient's neck.

35. A rescue system, comprising:
a pressure responsive valve system that is configured to permit respiratory gases to be passed through the valve system and to prevent the flow of respiratory gases through the valve system until a threshold negative intrathoracic pressure is exceeded;
a facial mask that is adapted to be placed over the patient's mouth and nose, wherein the valve system is adapted to be coupled to the facial mask; and
a communications device that is adapted to provide at least one way communication signals to an emergency center system.

36. A system as in claim 35, wherein the communications device is adapted to send communication signals to a transceiver, and wherein the transceiver is configured to establish a communication link with the emergency system.

37. A system as in claim 36, wherein the transceiver includes a processor and memory having stored information about a patient, and wherein the transceiver is configured to transmit the stored information to the emergency system.

38. A system as in claim 37, wherein the transceiver is configured to communicate with the emergency system by calling a 911 emergency number.

39. A system as in claim 35, wherein the communications device comprises a transceiver that is configured to establish a two way communications link with the emergency center system.

40. A system as in claim 39, wherein the transceiver includes a processor and memory having stored information about a patient, and wherein the transceiver is configured to transmit the stored information to the emergency system.

41. A system as in claim 40, wherein the transceiver is configured to communicate with the emergency system by calling a 911 emergency number.

42. A system as in claim 35, further comprising a head rest that is adapted to be placed under a patient's neck to elevate and tilt the patient's head back away from the patient's chest to assist in ventilating the patient.

43. A system as in claim 42, wherein the head rest includes a compartment for holding the communications device.

44. A system as in claim 43, wherein the head rest further includes a sensor to sense access to the compartment and to send a signal to actuate the communications device.

45. A method for rescuing a patient, the method comprising:
actuating a communications device that is programmed to automatically call an emergency service system;
coupling a facial mask over the patient's mouth and nose and to the head rest, wherein the facial mask includes a pressure responsive valve system that is configured to permit respiratory gases to be actively passed through the valve system and to prevent the flow of respiratory gases through the valve system until a threshold negative intrathoracic pressure is exceeded; and
repeatedly compressing the patient's chest.

46. A method as in claim 45, wherein the communications device is further programmed with information about a patient and to automatically transmit the patient information to the emergency service system.

47. A method as in claim 45, wherein the communications device is programmed to transmit a signal to a transceiver, and wherein the emergency service system is called using the transceiver to establish a communications link with the emergency service system.

48. A method as in claim 45, further comprising placing a head rest under the patient's neck to elevate and tilt the patient's head back away from the patient's chest to assist in ventilating the patient.

49. A method as in claim 45, wherein the communications device is stored in the head rest having a compartment, and wherein the communications device is actuated upon access to the compartment.

50. A method as in claim 45, further comprising periodically forcing respiratory gases through the valve system to ventilate the patient.

51. A method as in claim 48, further comprising coupling the head rest to the facial mask with a strap.

52. A method as in claim 48, further comprising accessing the compartment and removing the valve system and the facial mask from the compartment.

53. A method as in claim 48, further comprising actuating an electronic metronome that is stored in the compartment.

54. A method as in claim 48, further comprising providing a cooling package in the head rest, and using the cooling package to lower the temperature of the head rest.

55. A method as in claim 48, wherein the head rest is part of a cervical spine neck stabilizer.
